(19) United States
(12) Patent Application Publication
(10) Pub. No.: US 2012/0034626 A1
(43) Pub. Date: Feb. 9, 2012

(54) Neoplasia-Specific Autoantibodies and Methods

(76) Inventors: D. James Morré, West Lafayette, IN (US); Xiaoyu Tang, West Lafayette, IN (US)

(21) Appl. No.: 13/040,232

(22) Filed: Mar. 3, 2011

Related U.S. Application Data
(60) Provisional application No. 61/310,113, filed on Mar. 3, 2010.

(51) Int. Cl. G01N 33/577; C07K 19/00; G01N 33/573; C07K 16/40

(52) U.S. Cl. 435/7.23; 530/389.7; 530/387.3; 435/7.4

(57) ABSTRACT

ENOX2 proteins are growth-related cell surface proteins expressed specifically by cancer cells; they catalyze NADH oxidation and protein disulfide-thiol interchange reactions. Taught herein are IgM class autoantibodies specific to ENOX2 (tNOX) in a variety of cancer patient sera. Early cancer patients produce these autoantibodies as a possible defense mechanism. Because ENOX2 is bound to autoantibodies in patients, it is unavailable to bind conventional ENOX2-specific antibodies in standard ELISA assays, but two-dimensional gel electrophoresis dissociates ENOX2 protein from autoantibodies, allowing detection. Probing ENOX2 using cancer sera as a source of ENOX2 autoantibodies followed by horseradish peroxidase-coupled anti-human IgM allows visualization and detection of the ENOX2 autoantibody. ENOX2 autoantibodies from breast cancer sera reacts with the ENOX2 isoforms from, e.g., lung and ovarian cancer patient sera. ENOX2 autoantibodies enable cancer screening based both on autoantibody detection and autoantibody dissociation to allow for standard ELISA development as well as therapy.

### CROSS-REFERENCE TO RELATED APPLICATIONS

This application claims benefit of U.S. Provisional Application No. 61/310,113, filed Mar. 3, 2010, which application is incorporated by reference herein to the extent there is no inconsistency with the present disclosure.

### REFERENCE TO SEQUENCE LISTING

The Sequence Listing submitted herewith is incorporated by reference herein.

### BACKGROUND

The field of this invention is the area of cancer immunology, in particular, as related to the diagnosis of neoplastic cells, as specifically related to patient-generated autoantibodies specific to ENOX2 (tNOX) a cell surface marker characteristic of neoplasia in general and isoforms which exhibit specific patterns of protein expression indicative of specific types of cancer. Detection of the autoantibodies is by use of specific antibodies to the autoantibodies (AuAbAb).

ENOX2 is a cancer-specific member of a unique, growth-related family of cell surface hydroquinone or NADH oxidases with protein disulfide-thiol interchange activity referred to as ECTO-NOX or ENOX proteins (for cell surface NADH oxidases) (More, 1998, in Plasma Membrane Redox Systems and Their Role in Biological Stress and Disease, H. Asard, A. Bérczi and R. J. Caubergs, eds., pp. 121-156, Kluwer Academic Publishers, Dordrecht, Netherlands; Morré and Morré. Free Radical Press 37: 7905-808). ENOX2 (or tNOX for tumor associated) is specific to the surfaces of cancer cells and the sera of cancer patients (Morré et al. 1995, Proc. Natl. Acad. Sci. USA 91: 1831-1835; Bruno et al. 1992, Biochem. J. 281: 625-628). The presence of the ENOX2 protein has been demonstrated for several human tumor tissues (mammary carcinoma, prostate cancer, neuroblastoma, colon carcinoma and melanoma) (Cho et al. 2002, Cancer Immunol. Immunother. 51: 121-129). Serum analyses suggest a much broader association with human cancer (Morré et al. 1997, Arch. Biochem. Biophys. 342: 224-230; Morré and Reust, 1997, J. Bioenerg. Biomemb. 29: 281-289).

ENOX proteins are ectoproteins anchored in the outer leaflet of the plasma membrane (Morré, 1995, Biochim. Biophys. Acta 1240: 201-208). As is characteristic of other examples of ectoproteins (sialyl and galactosyl transferase, dipeptidylamino peptidase IV, etc.), the ENOX proteins are shed. They appear in soluble form in conditioned media of cultured cells (Cho et al. 2002, Cancer Immunol. Immunother. 51: 121-129) and in patient sera (Morré et al. 1997, Arch. Biochem. Biophys. 342: 224-230; Morré and Reust, 1997, J. Bioenerg. Biomemb. 29: 281-289). The serum form of ENOX2 from cancer patients exhibits the same degree of drug responsiveness as does the membrane-associated form. Drug-responsive ENOX2 activities are seen in sera of a variety of human cancer patients, including patients with leukemia, lymphomas or solid tumors (prostate, breast, colon, lung, pancreas, ovarian, liver) (Morré et al. 1997, Arch. Biochem. Biophys. 342: 224-230; Morré and Reust, 1997, J. Bioenerg. Biomemb. 29: 281-289). An extreme stability and protease resistance of the ENOX2 protein (del Castillo-Olivares et al. 1998, Arch. Biochem. Biophys. 358: 125-140) may help explain its ability to accumulate in sera of cancer patients to readily detectable levels. In contrast, no drug-responsive ENOX activities have been found in the sera of healthy volunteers (Morré et al. 1997, Arch. Biochem. Biophys. 342: 224-230; Morré and Reust, 1997, J. Bioenerg. Biomemb. 29: 281-289) or in the sera of patients with disorders other than cancer.

Because cancer poses a significant threat to human health and because cancer results in significant economic costs, there is a long-felt need in the art for an effective, economical and technically simple system in which to assay for the presence of cancer.

### SUMMARY

Provided herein is a method for the analysis of a biological sample for the presence of patient-generated autoantibodies to ENOX2, for example, in methods for diagnosis of cancer in a patient suspect of having cancer (or other neoplastic disease). The present method entails isolation of an IgM fraction from cancer patient sera containing the autoantibodies as antigen for generation of cancer specific anti-autoantibody antibodies (AuAbAb) in mice followed by clonal selection to generate autoantibody antibodies specific to autoantibodies for the pan-cancer ENOX2 antigen and its various isoforms which characterize particular types of cancers. Alternatively, the autoantibody from patient sera or generated in bacteria (or other recombinant cell type, including but not limited to, mammalian, yeast or fungal cells) as a single-chain variable region antibody fragment (single chain autoantibody or recombinant antibody or equivalent) is used as a primary antibody. All of the above may be adapted to either a western blot or ELISA format. As specifically exemplified the single chain antibody with the specificity of the autoantibody specific to tNOX from cancer patients is characterized by the sequence set forth in SEQ ID NO:6, amino acids 1-297 or 1-312.

Further provided are IgM heavy and light chain autoantibody proteins with specificity for the cancer-specific ENOX2 antigen(s). Coding and amino acid sequences for the heavy and light chains are given in Tables 4 and 5 and in SEQ ID NOs:7, 9, 8 and 10, respectively. Isolated antibodies specific to ENOX2 can be purified from cancer serum using ENOX2 or recombinantly expressed ENOX2 as an affinity ligand, for example, or any other means known to the art.

An additional embodiment is a method for treating a cancer, said method comprising administering to a cancer patient in need thereof an effective amount of a pharmaceutical composition comprising the ENOX2-specific IgM autoantibody or recombinant single chain antibody described herein. A specifically exemplified IgM can have amino acid sequences for the heavy and light chains as given in Tables 4 and 5 and in SEQ ID NOs:8 and 10 or a single chain “autoantibody” as set forth SEQ ID NO:6, amino acids 1-297. It is understood that the particular sequences of the heavy and light chains may vary from those specifically exemplified herein but the binding site of the IgM antibody retains the specificity for the ENOX2 protein. Such an antibody can be conjugated to an anticancer agent, as well.

Another embodiment herein is a single chain antibody with specificity for the ENOX2-specific autoantibody produced by cancer patients (described above). The coding and amino acid sequences of the single chain antibody are given in SEQ ID NO:5 and 6, respectively. This single chain antibody can be employed in assays of biological samples from cancer patients, especially sera or biopsy tissue, but also including but not limited to urine, peritoneal fluid, blood, cerebrospinal fluid. Reactivity with the ENOX2-specific IgM autoantibody denotes the presence of cancer in the patient from whom the biological sample was taken. Detection of the reactivity can be via a western blot or an ELISA format.

Further provided herein are methods for the detection of presence of cancer and ultimately of the cell type or tissue of cancer origin (breast, ovarian prostate, etc.) in simple direct or sandwich ELISA formats. At present there are no other pan cancer (all forms of human cancer) tests with this particular capability.

Also provided are methods for determining neoplasia in a mammal, including a human, said method comprising the steps of detecting cancer presence, in a biological sample. The present disclosure further enables obtaining additional information for assessment of neoplasia, including a measure of tumor burden, for example, in serum, plasma or in biopsy material based on levels of fully processed 34 kDa ENOX2 (among certain other isoforms of ENOX2) as detected and/or quantitated using either the natural autoantibodies or recombinant autoantibodies.

Also within the scope of the present disclosure are anti-autoantibody or autoantibody detection of particular isoforms of ENOX2 associated with specific (primary) cancers. Positive results are indicative of the presence of cancer, and the detection of characteristic autoantibodies may allow a presumption as to the primary incidence of cancer in that patient according to the association of particular autoantibodies to ENOX2 proteins associated with particular cancer origins, as set forth above.

The methods provided herein can also be applied to evaluate response to therapy, with decreasing amounts of fully processed ca. 34 kDa ENOX2 as detected either by natural autoantibodies or recombinant autoantibodies reflecting successful treatment, as well as early detection of recurrent disease as reflected by increased or reappearance of ENOX2-specific isoforms or autoantibodies using ELISA-based detection technology.

### BRIEF DESCRIPTION OF THE FIGURES

FIG. 1 provides the results of analytical 2-D gel electrophoresis of sera from a breast cancer patient. Separation in the first dimension was by isoelectric focusing and in the second dimension by SDS-PAGE (using ampholytes over the pH range of 3 to 10 and 10% polyacrylamide gel). Detection was with IgM class autoantibodies present in sera of the same patient and alkaline phosphatase-linked anti-IgM as the secondary detecting antibody. Only quadrant IV of the gel is depicted. Shown is the 68 kDa, isoelectric point pH 4.5 ENOX2 isoform specific for breast cancer. Ab=location of serum albumin.

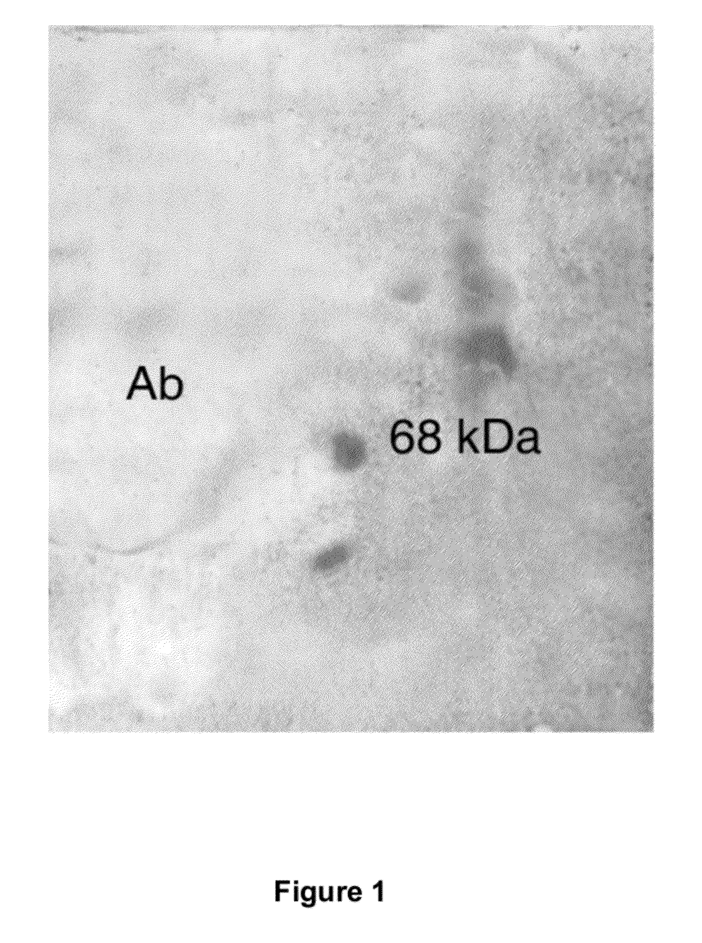

FIG. 2 is as in FIG. 1, except detection was with recombinant anti-tNOX single chain variable region autoantibody to ENOX2 generated in bacteria and carrying an S-tag followed by alkaline phosphatase-linked anti S (Novagen cat. #69598-3 or equivalent product) with Western Blue NBT substrate (Promega, Madison, Wis.; Cat. No. S3841 or equivalent product). The recombinant autoantibody originated from B cells of an ovarian cancer patient. Shown is its ability to react with the 80 kDa, isoelectric point pH 4.2 ENOX2 ovarian cancer-specific isoform from sera of the same patient. The gel was patient sera. The ELISA was a lung cancer-specific monoclone.

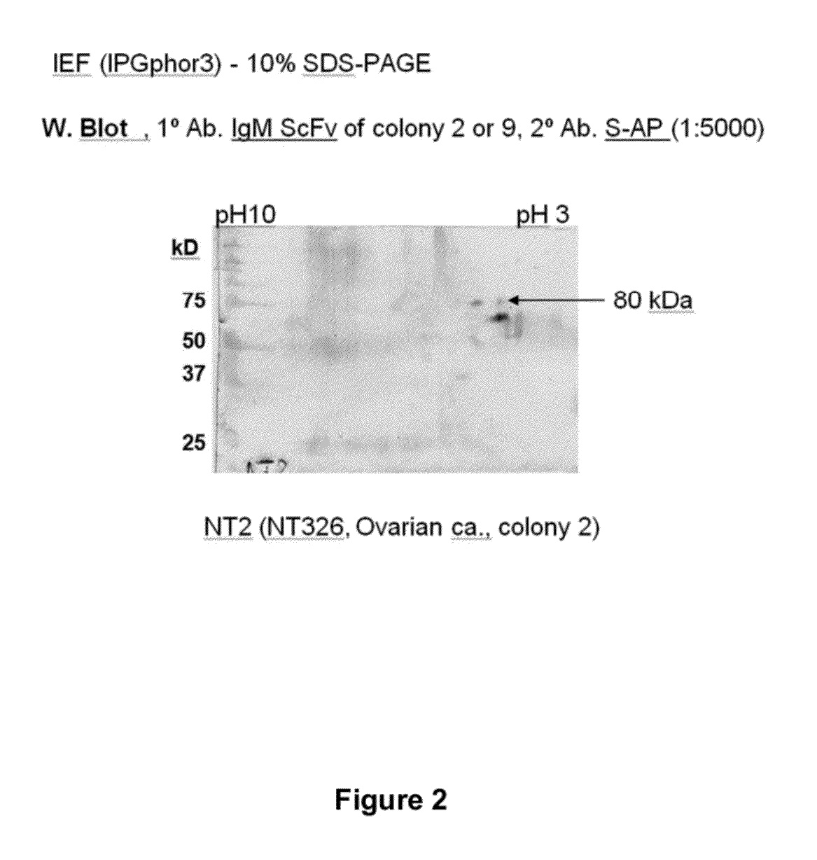

FIG. 3 shows a general scheme for an ELISA based analysis of cancer presence based on the use of antibodies (AuAbAb) specific to the autoantibody.

FIG. 4 shows 2D-polyacrylamide gel electrophoresis western blots developed with sera of different cancer patients as ENOX2 antigen source and pooled sera from breast cancer patients as source of autoantigen. The tumor-specific ENOX2 transcription variants are indicated by single arrows. The 33 to 38 kDa fully processed remnant uniquely revealed by the endogeous autoantibody is indicated by double arrows. (See also Table 6).

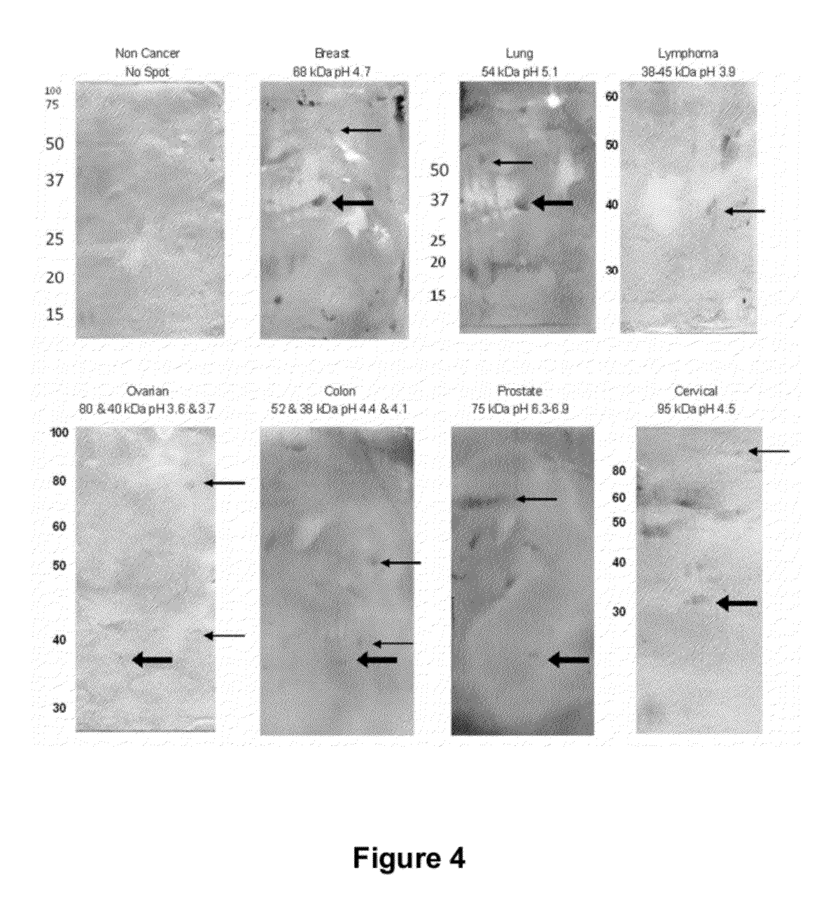

### DETAILED DESCRIPTION

Described herein are patient-generated autoantibodies of the IgM class to cancer specific ENOX2 and its isoforms present in human sera, which isoforms are indicative of cancer presence, tumor type, disease severity and therapeutic response. The autoantibodies themselves or antibodies generated in mice to the autoantibodies may be used in an ELISA format or in conjunction with an isoform-resolving two-dimensional gel electrophoresis protocol and subsequent immunoanalysis to detect tNOX isoforms which are characteristic of particular cancers.

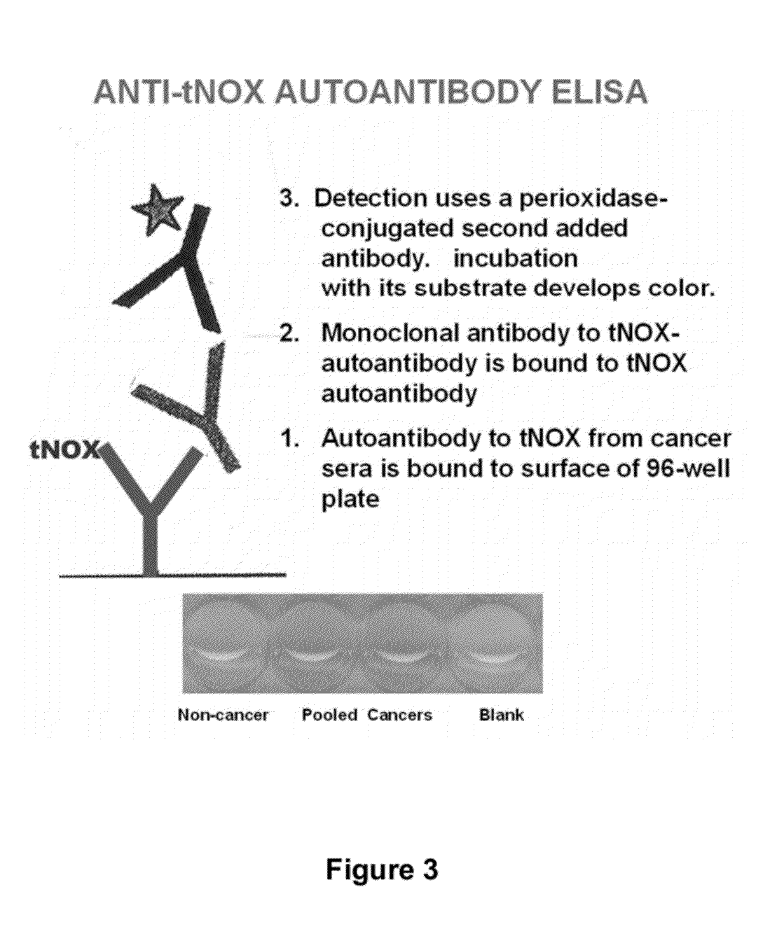

For ELISA detection, proteins from cancer sera are absorbed to wells of a 96 well plate and the detecting antibodies are added sequentially as illustrated in FIG. 3. For western blot analysis, the isoforms are blotted onto a nitrocellulose membrane for further analysis using the autoantibody preparations.

For generation of antibodies specific to the ENOX2 autoantibodies, rabbits were immunized and sera were prepared against an IgM fraction isolated from cancer patient sera. Monoclones were then selected from hybridomas derived from spleen cells of the immunized mice by screening against cancer patient sera using an ELISA-based protocol (see herein below). Clones having no observed reactivity with sera from non-cancer patients were selected. Antibody specific to the ENOX2-specific autoantibodies are not reactive with all IgM antibodies.

Autoantibodies specific for the plasma membrane ENOX2 isoforms and their circulating counterparts in sera and other body fluids of cancer patients and animals with neoplastic disorders are useful, for example, as probes for detecting or diagnosing cancer or a neoplastic disorder in a sample from a human or animal. The (detectable) anti-autoantibodies which are specific for the autoantibody which recognizes ENOX2 or the ENOX2-specific autoantibodies) can be bound to a substance which provides a cofactor, inhibitor, fluorescent agent, chemiluminescent agent, magnetic particle, radioisotope or other detectable signal. Suitable labels include but are not limited to radionuclides, enzymes, substrates, magnetic particles and the like. United States patents describing the use of such detectable moieties (labels) include, but are not limited to, U.S. Pat. Nos. 3,817,837; 3,850,752; 3,939,350; 3,996,345; 4,277,437; 4,275,149; 4,331,647; 4,348,376; 4,361,544; 5,444,744; 4,460,561; 4,624,846; 4,366,241, 5,716,595; among others. For use in therapeutic regimens, the antibody provided herein can be coupled to a therapeutic radionuclide, a chemotherapeutic agent, a ribonucleolytic agent or a toxin. See, among others, U.S. Pat. Nos. 5,541,297, 6,395,276. The invention may be further understood by the following non-limiting examples.

### EXAMPLES

### Example 1

### Experiments with Pooled Sera

ENOX2 isoform proteins from sera pooled from cancer patients (breast, ovarian, lung and colon) were resolved by 2-D gel electrophoresis, with detection by ENOX2 autoantibody from patient sera and followed by alkaline phosphatase-linked anti-IgM with Western Blue NBT alkaline phosphatase substrate. The ENOX2-specific autoantibodies recognized several proteins present in the cancer sera but absent in sera of non-cancer patients or healthy volunteers. Examples were ENOX2 isoforms characteristic of breast, lung and ovarian cancer.

### Example 2

### Analysis of Sera from Patients with Various Cancers

ENOX2 from sera of a breast cancer patient (Mr 68 kDa, isoelectric point 4.5) was uniquely detected on western blots by autoantibodies from a breast cancer patient.

### Example 3

### Analysis of Cervical Carcinoma (HeLa) Cells in Culture

2-D gel analysis when applied to cervical carcinoma (HeLa) cells yield the cervical carcinoma ENOX2 isoform of about 98 kDa and isoelectric point 4.0 on western blots detected using autoantibodies from a breast cancer patient.

### Example 4

### Recombinant Autoantibody Analysis of ENOX2 Isoforms in Pooled Sera of Cancer Patients

Experiments were carried out as in Example 1 except that natural autoantibodies were replaced with single chain recombinant autoantibodies produced in bacteria.

### Example 5

### Recombinant Autoantibody Analysis of Sera of Patients with Various Cancers

Experiments were carried out as in Example 2 except autoantibodies were single chain recombinant autoantibodies produced in bacteria.

### Example 6

### Analysis of Patient Sera Using Monoclonal Antibodies specific to the ENOX2 Autoantibody from an Ovarian Cancer Patient

Results from an ELISA assay are illustrated in Table 1.

TABLE 1
ANTI-tNOX AUTOANTIBODY ELISA

| | ABSORBANCE - BLANK |
|---|---|
| Non-cancer | 0.080 ± 0.035 (>0.150) |
| Ovarian Cancer - | 0.435 ± 0.064 |
| Pooled | |
| Ovarian Cancer - | 0.373 ± 0.019 |
| Individual | |
| Lung Cancer - | 0.303 ± 0.024 |
| Pooled | |
| Breast Cancer - | 0.357 ± 0.048 |
| Pooled | |

### Example 7

### Analysis of Patient Sera Using a Monoclonal Antibody to the ENOX2 Autoantibody from an Ovarian Cancer Patient Specific for the Lung Cancer-Specific ENOX2 Isoform

Results from an ELISA assay are illustrated in Table 2.

TABLE 2
LUNG CANCER-SPECIFIC ANTI-tNOX AUTOANTIBODY
(AuAbAb) ELISA*

| | ELISA absorbance-Blank |
|---|---|
| Non-Cancer Pooled - | 0.39 ± 0.08 |
| Individual | 0.34 ± 0.08 |
| | >0.53 |
| Lung Cancer Pooled | 0.72 ± 0.02 |
| GHS 89671 (NSC) | 0.66 ± 0.05 |
| GHS 41457 (SCL) | 0.69 ± 0.08 |
| Breast Cancer Pooled | 0.45 ± 0.05 |
| GHS 33357 | 0.46 ± 0.05 |
| GHS 56741 | 0.51 ± 0.02 |
| Ovarian Cancer Pooled | 0.38 ± 0.06 |
| NT 326 | 0.30 ± 0.02 |

*In roller bottle production

### Example 8

### Analysis of Patient Sera Using a Monoclonal Antibody Specific to the ENOX2 Autoantibody from a Lung Cancer Patient

Experiments were carried out as in Examples 6 and 7 except that antibodies to the autoantibodies of a lung cancer patient were used.

### Example 9

### Analysis of Patient Sera Using a Monoclonal Antibody Specific to the ENOX2 Autoantibody from a Breast Cancer Patient

Experiments were carried out as in Examples 6 and 7 except antibodies raised against the autoantibodies of a breast cancer patient were used.

### Example 10

### ENOX2-Specific Autoantibodies are IgM Class

The ENOX2-specific autoantibodies are detected exclusively with anti IgM-specific antisera.

### Example 11

### Analysis of Non-Cancer Sera

In more than 25 randomly selected outpatient sera and sera of healthy volunteers, no ENOX2-specific autoantibodies were detected, confirming previous observations that ENOX2 proteins are absent from non-cancer patients or sera of healthy volunteers and that ENOX-2-specific antibodies are not present in healthy persons.

### Example 12

### Recombinant Autoantibody Production

For a recombinant autoantibody, cDNAs encoding the variable regions of immunoglobulin heavy chain (VH) and light chain (VL), were cloned by using degenerate primers. Mammalian immunoglobulins of light and heavy chain contain conserved regions adjacent to the hypervariable complementary defining regions (CDRs). Degenerate oligoprimer sets allow these regions to be amplified using PCR (Jones et al. 1991. Bio/Technology 9:88-89; Daugherty et al. 1991. Nucl. Acids Res. 19:2471-2476). Recombinant DNA techniques have facilitated the stabilization of variable fragments by covalently linking the two fragments by a polypeptide linker (Huston et al. 1988. Proc. Natl. Acad. Sci. USA 85:5879-5883). Either V_{L} or V_{H} can provide the NH₂-terminal domain of the single chain variable fragment. The linker should be designed to resist proteolysis and to minimize protein aggregation. Linker length and sequences contribute and control flexibility and interaction with recombinant single chain recombinant autoantibody and antigen. The most widely used linkers have sequences consisting of glycine (Gly) and serine (Ser) residues for flexibility, with charged residues as glutamic acid (Glu) and lysine (Lys) for solubility (Bird et al. 1988. Science 242:423-426; Huston et al. 1988. supra).

Isolation of lymphocytes. Ten ml of blood was collected into purple top BD Vacutainer tubes containing K2EDTA from an ovarian cancer patient. Isolation of lymphocytes was performed according to the procedure of Ficoll-Paque™ Plus (density gradient centrifugation medium) instruction (71-7167-00AG, GE Healthcare, Waukesha, Wis.). To two 3 ml of Ficoll-Paque™ Plus aliquots, 4 ml of blood was carefully layered into each aliquot. Samples were centrifuged at 400×g for 40 min in a swinging bucket rotor. Plasma was removed and lymphocytes were carefully collected to 3 ml, diluted to 14 ml with Balanced Salt solution (D-glucose 0.01%, calcium chloride 5 μM, magnesium chloride 98 μM, potassium chloride 0.54 mM, Tris 14.5 mM and sodium chloride 126 mM) and centrifuged at 400×g for 10 min. Supernatant was removed and lymphocytes were resuspended in 5 ml of separation buffer (phosphate buffered solution Na₂HPO₄*2 H₂O, 8.1 mM; KH₂PO₄, 1.76 mM; NaCl, 137 mM; and KCl; 2.7 mM, pH 7.4, 2% fetal bovine serum; 1 mM EDTA).

Isolation of B lymphocytes producing IgM. One mg of DNAse I (StemCell Technologies, Vancouver, CA) was added to the lymphocyte cells and incubated at room temperature for 15 min. Cells were passed through 70 μm mesh nylon strainer. 35 ml of separation buffer was added and centrifuged at 1000×g for 50 min in a swinging bucket rotor without deceleration brake. Supernatant was decanted and pellet was resuspended in 2 ml of separation buffer in 5-ml FalconR polystyrene round bottom tube. Isolation of B lymphocytes producing IgM was performed according to the instruction provided by the EasySep PE Selection Kit (StemCell Technologies). 200 μl of FcR blocking antibody for human cells was added. 200 μl of phycoerythrin-conjugated antibody was added and mixed well. The cells were incubated at room temperature for 15 min. 200 μl of EasySepR Magnetic Nanoparticles was added and cells were incubated at room temperature for 10 min. Tube was placed into the EasySepR magnet for 5 min. Tube was inverted and supernatant was decanted. Tube was removed from the magnet and cells were resuspended in 2.5 ml of separation buffer. Cells were washed two more times by using the magnet. After washing, cells were resuspended in 2.5 ml of separation buffer for cDNA isolation.

mRNA was purified from the isolated IgM-producing B lymphocytes using Oligotex direct mRNA mini kit (Qiagen). Preheated Elution buffer (70° C., 100 μl) was applied to the spin column, and mRNA was eluted and collected in 1.5 ml of microcentrifuge tubes on ice.

Reverse transcription was carried out using M-MLV reverse transcriptase. For each 50 μl of cDNA synthesis, 2 μl L random primers and 24.7 μl of mRNA were mixed and incubated at 70° C. for 5 min, then put on ice for 2 min. Then, 10 μl M-MLV buffer (5×), 10 μl dNTP mix, 1.3 μl of Rnasin (40 u/μl) and 2 μl of M-MLV reverse transcriptase were added. The mixture was incubated for 120 min at 37° C.

Four rounds of PCR were performed to get final products to be inserted into the pET11a vector. First round of PCR was performed to get IgM heavy chain and light chain. Degenerate primers for light chain and heavy chain are: VH-FOR (Heavy Chain Forward): SAG GTG MAG YTG KTG GAG TCT GG (S=C,G; M=A,C; Y=C,T; K=G,T), SEQ ID NO:14; CHE-REV (Heavy Chain Reverse): AAG TGA TGG AGT CGG GAA GGA AGT, SEQ ID NO:15; HK-FOR (Light Chain Forward): GAA ATW GTR WTG ACR CAG TCT CCA (W=A,T; R=A,G), SEQ ID NO:16; CK-REV (Light Chain Reverse): GAT GAA GAC AGA TGG TGC AGC CAC, SEQ ID NO:17. Second round of PCR was performed to add linker to Heavy chain 3′ end and light chain 5′ end. Primers used to add linker to Heavy chain are: heavy #3: ATA TCA GCC GGC CSA GGT GMA GYT GKT G (degenerate S=C, G; M=A,C; Y=C,T; K=G,T), SEQ ID NO:18; heavy #4: AGA GCC GCC GCC ACC CGA GCC GCC ACC GCC CGA TCC ACC GCC TCC GAA AGT GAT GGA GTC GG, SEQ ID NO:19; Primers used to add linker to Light chain are: Light #1: GGA GGC GGT GGA TCG GGC GGT GGC GGC TCG GGT GGC GGC GGC TCT GAA ATW GTR WTG ACR CAG TCT CCA (degenerate W=A,T; R=A,G), SEQ ID NO:20; light #2: AGC GGC CGC GAT GAA GAO AGA TGG TGC AGC CAC, SEQ ID NO:21. Then, heavy #3 and light #2 primers were used to run the third round of PCR to get Heavy chain-linker-light chain product. The purpose of the final round of PCR was to add restriction sites (NheI and BamHI) and S-tag. Primers used are NheI-Hvy3Fr1: CAT ATA CAT ATG GCT AGC AAT CAG CCG GCC SAG GT (S=C,G), SEQ ID NO:22; and L2S-BamHI: TAG CAT CCG GAT CCT TAA CTG TCC ATG TGC TGG CGT TCG AAT TTA GCA GCT GCG GTT TCT TTG ATG AAG ACA GAT GGT GC, SEQ ID NO:23. PCR synthesis was carried out in 50 μl reaction volumes in 0.2 ml microcentrifuge tubes by using Mastercycler pro (Eppendorf). All PCR synthesis included 0.5 μl o forward and reverse primers (50 μM), 4 μl of cDNA or PCR products as template, 1 μl of 10 mM dNTPs, 10 μl of 10× pfu buffer, 1 μl of polymerase (pfu polymerase 0.9 μl and Taq polymerase 0.1 μl). All PCR profiles consisted of one cycle of 2 min of denaturation at 94° C.; 30 cycles of 45 sec of denaturation at 94° C., 45 sec of annealing at 55° C. and 2 min of extension at 72° C.; and one cycle of 10 min of extension at 72° C. PCR products were purified with Qiaex II gel extraction kit from Qiagen. PCR amplification products were analyzed by agarose gel electrophoresis.

The DNA sequence encoding the linker was 45 nucleotides long (GGAGGCGGTGGATCGGGCGGTGGCGGCTCGGGTGGCGGCGGCTCT; SEQ ID NO:1), which translates to a peptide of 15 amino acids (GlyGlyGlyGlySerGlyGlyGlyGlySerGlyGlyGlyGlySer; SEQ ID NO:2). S-peptide was linked to the C-terminus of recombinant single chain autoantibody, ScFv(S). S-peptide binds to S-protein conjugated to alkaline phosphatase for Western blot analysis. The DNA sequence of the S-peptide is AAAGAAACCGCTGCTGCTAAATTCGAACGCCAGCACATGGACAGC (SEQ ID NO:3) which translates to S-peptide (LysGluThrAlaAlaAlaLysPheGluArgGln HisMetAspSer; SEQ ID NO:4).

Plasmid pET11a and purified PCR products were digested with restriction enzymes NheI and BamHI and ligated to produce plasmid pET11a-scFv(S). E. coli BL21 (DE3) was transformed with pET11a-scFv(S). Positive colonies were selected by colony PCR. Primers used are NheI-Hvy3Fr1: CAT ATA CAT ATG GCT AGC AAT CAG CCG GCC SAG GT (S=C,G), SEQ ID NO:23; and L2S-BamHI: TAG CAT CCG GAT CCT TAA CTG TCC ATG TGC TGG CGT TCG AAT TTA GCA GCT GCG GTT TCT TTG ATG AAG ACA GAT GGT GC, SEQ ID NO:24. Individual colony was picked and added to 20 μl of 0.1× Taq buffer. After 5 min at 100° C., the samples were centrifuged for 5 min at 13,000 rpm. Five μl of the supernatant was used as template for PCR (total PCR volume was 20 μl). Positive colonies were grown at 37° C. for 12 h in LB medium containing ampicillin (100 μg/ml). Expression of the recombinant single chain autoantibody was induced by addition of 1 mM IPTG and incubation for 4 h. A small volume of cells (less than 10 ml) was harvested, and the cells were lysed using a CelLytic B Plus Kit (Sigma-Aldrich, St. Louis, Mo.). Expression of recombinant single chain autoantibody was tested by Western blot analysis using S-AP (S-protein conjugated to alkaline phosphatase) as antibody. For large volume (200 ml) of cells, cells were lysed using a French Pressure Cell (French Pressure Cell Press, SLM Instruments, Inc.) (three passages at 20,000 psi). Cell extracts were centrifuged at 10,000×g for 20 min. Pellets containing inclusion bodies of recombinant single chain autoantibody were collected. Renaturation of the inclusion bodies of the recombinant single chain autoantibody was according to Goldberg et al. (1995) Folding and Design 1; 21-27.

The coding and amino acid sequences characterizing the recombinant single chain autoantibody with human sequence and specificity for cancer-specific ENOX2 is given in Table 3 and in SEQ ID NOs:5 and 6, respectively. The theoretical pl/Mw (average) for the recombinant single chain autoantibody disclosed herein is 7.57/32911.73. A summary of this single chain molecule is as follows: Fv heavy chain 164aa, linker 15aa, light chain 118 amino acids, S-tag: 15 amino acids Total: 312 amino acids. The total coding sequence is 936 nucleotides in length, followed by a translation stop codon.

TABLE 3
Aligned Coding and Amino Acid Sequences of
single chain autoantibody (SEQ ID NOs: 5 and 6,
respectively). The coding sequence extends from
nucleotide 1 through 936, excluding the
translation termination codon, in SEQ ID NO: 5.

```
atggctagcaatcagccggcagaggtgcagctggtggagtctggggct
  M A S N Q P A E V Q L V E S G A
gaggtgaagaagcctggggcctcagtgaaggtttcctgcaaggcttct
  E V K K P G A S V K V S C K A S
ggatacaccttcactagctatgctatgcattgggtgcgccaggccccc
  G Y T F T S Y A M H W V R Q A P
ggacaaaggcttgagtggatgggatggatcagcgcttacaatggtaac
  G Q R L E W M G W I S A Y N G N
acaaactatgcacagaagctccagggcagagtcaccatgaccacagtc
  T N Y A Q K L Q G R V T M T T V
acatccacgagcacagcctacatggagctgaggagcctgagatctgac
  T S T S T A Y M E L R S L R S D
ggcacggccgtgtattactgtgcgagagatctgctagtgggcctcggc
  G T A V Y Y C A R D L L V G L G
tactggggccagggaaccctggtcaccgtctcctcagggagtgcatcc
  Y W G Q G T L V T V S S G S A S
gccccaacccttttccccctcgtctcctgtgagaattccccgtcggat
  A P T L F P L V S C E N S P S D
acgagcagcgtggccgttggctgcctcgcacaggacttccttcccgac
  T S S V A V G C L A Q D F L P D
tccatcactttcggaggcggtggatcgggcggtggcggctcgggtggc
  S I T F G G G G S G G G G S G G
ggcggctctgaaatagtattgacacagtctccagccaccctgtctgtg
  G G S E I V L T Q S P A T L S V
tctccaggggaaagagccaccctctcctgcagggccagtcagagtgtt
  S P G E R A T L S C R A S Q S V
agcagccacttagcctggtaccaacagaaacctggccaggctcccagg
  S S H L A W Y Q Q K P G Q A P R
ctcctcatctatgatgcatccaacagggccactggcatcccagacagg
  L L I Y D A S N R A T G I P D R
ttcagtggcagtgggtctgggacagacttcactctcaccatcagcaga
  F S G S G S G T D F T L T I S R
ctggagcctgaagattttgcagtgtattactgtcagcagtatggtagc
  L E P E D F A V Y Y C Q Q Y G S
tcacctccgtacactttcggccctgggaccaaagtggatatcaaacga
  S P P Y T F G P G T K V D I K R
actgtggctgcaccatctgtcttcatcaaagaaaccgcagctgctaaa
  T V A A P S V F I K E T A A A K
ttcgaacgccagcacatggacagttaa
  F E R Q H M D S -
```

Since the recombinant single chain antibody herein is monovalent and small in size, its functional affinity can be improved through multimerization (Albrecht et al. (2006) Mono specific bivalent scFv-SH: Effects of linker length and location of an engineered cysteine on production, antigen binding activity and free SH accessibility. J. Immunol. Meth. 310:100-116). Thus, the present scFv has been modified by increasing the joining linker length for higher production and better antigen binding as described by Albrecht et al. (2006). A 20 aa long linker (G4S)₄ was the longest linker tested. The free thiol introduced at the C terminal end of a scFv (scFv-SH) allows for site-specific covalent attachment to a PEG scaffold. In certain experiments, a Divalent recombinant single chain autoantibody with four repeats of the flexible linker unit G4S (scFv-G4S G4S G4S G4S-scFv) was used.

TABLE 4
Aligned Coding and Amino Acid Sequences of the
IgM Autoantibody Heavy Chain Coding and Protein
Sequences (SEQ ID NOs: 7 and 8, respectively).
The coding sequence extends from nucleotide 32
through 1828, excluding the translation
termination codon, in SEQ ID NO: 7.

```
ttttgtttaactttaagaaggagatatacatatggctagcaatcagcc
                                                              M A S N Q P
ggcagaggtgcagctggtggagtctggggctgaggtgaagaagcctgg
  A E V Q L V E S G A E V K K P G
ggcctcagtgaaggtttcctgcaaggcttctggatacaccttcactag
    A S V K V S C K A S G Y T F T S
ctatgctatgcattgggtgcgccaggcccccggacaaaggcttgagtg
    Y A M H W V R Q A P G Q R L E W
gatgggatggatcagcgcttacaatggtaacacaaactatgcacagaa
    M G W I S A Y N G N T N Y A Q K
gctccagggcagagtcaccatgaccacagtcacatccacgagcacagc
    L Q G R V T M T T V T S T S T A
ctacatggagctgaggagcctgagatctgacggcacggccgtgtatta
    Y M E L R S L R S D G T A V Y Y
ctgtgcgagagatctgctagtgggcctcggctactggggccagggaac
    C A R D L L V G L G Y W G Q G T
cctggtcaccgtctcctcagggagtgcatccgccccaacccttttccc
    L V T V S S G S A S A P T L F P
cctcgtctcctgtgagaattccccgtcggatacgagcagcgtggccgt
    L V S C E N S P S D T S S V A V
tggctgcctcgcacaggacttccttcccgactccatcactttctcctg
    G C L A Q D F L P D S I T F S W
gaaatacaagaacaactctgacatcagcagcacccggggcttcccatc
    K Y K N N S D I S S T R G F P S
agtcctgagagggggcaagtacgcagccacctcacaggtgctgctgcc
    V L R G G K Y A A T S Q V L L P
ttccaaggacgtcatgcagggcacagacgaacacgtggtgtgcaaagt
    S K D V M Q G T D E H V V C K V
ccagcaccccaacggcaacaaagaaaagaacgtgcctcttccagtgat
    Q H P N G N K E K N V P L P V I
tgctgagctgcctcccaaagtgagcgtcttcgtcccaccccgcgacgg
    A E L P P K V S V F V P P R D G
cttcttcggcaacccccgcagcaagtccaagctcatctgccaggccac
    F F G N P R S K S K L I C Q A T
gggtttcagtccccggcagattcaggtgtcctggctgcgcgaggggaa
    G F S P R Q I Q V S W L R E G K
gcaggtggggtctggcgtcaccacggaccaggtgcaggctgaggccaa
    Q V G S G V T T D Q V Q A E A K
agagtctgggcccacgacctacaaggtgaccagcacactgaccatcaa
    E S G P T T Y K V T S T L T I K
agagagcgactggctcagccagagcatgttcacctgccgcgtggatca
    E S D W L S Q S M F T C R V D H
caggggcctgaccttccagcagaatgcgtcctccatgtgtgtccccga
    R G L T F Q Q N A S S M C V P D
tcaagacacagccatccgggtcttcgccatccccccatcctttgccag
    Q D T A I R V F A I P P S F A S
catcttcctcaccaagtccaccaagttgacctgcctggtcacagacct
    I F L T K S T K L T C L V T D L
gaccacctatgacagcgtgaccatctcctggacccgccagaatggcga
    T T Y D S V T I S W T R Q N G E
agctgtgaaaacccacaccaacatctccgagagccaccccaatgccac
    A V K T H T N I S E S H P N A T
tttcagcgccgtgggtgaggccagcatctgcgaggatgactggaattc
    F S A V G E A S I C E D D W N S
cggggagaggttcacgtgcaccgtgacccacacagacctgccctcgcc
    G E R F T C T V T H T D L P S P
actgaagcagaccatctcccggcccaagggggtggccctgcacaggcc
    L K Q T I S R P K G V A L H R P
cgatgtctacttgctgccaccagcccgggagcagctgaacctgcggga
    D V Y L L P P A R E Q L N L R E
gtcggccaccatcacgtgcctggtgacgggcttctctcccgcggacgt
    S A T I T C L V T G F S P A D V
cttcgtgcagtggatgcagagggggcagcccttgtccccggagaagta
    F V Q W M Q R G Q P L S P E K Y
tgtgaccagcgccccaatgcctgagccccaggccccaggccggtactt
    V T S A P M P E P Q A P G R Y F
cgcccacagcatcctgaccgtgtccgaagaggaatggaacacggggga
    A H S I L T V S E E E W N T G E
gacctacacctgcgtggtggcccatgaggccctgcccaacagggtcac
    T Y T C V V A H E A L P N R V T
cgagaggaccgtggacaagtccaccgagggggaggtgagcgccgacga
    E R T V D K S T E G E V S A D E
ggagggctttgagaacctgtgggccaccgcctccaccttcatcgtcct
    E G F E N L W A T A S T F I V L
cttcctcctgagcctcttctacagtaccaccgtcaccttgttcaaggt
    F L L S L F Y S T T V T L F K V
gaaatgatcccaacagaagaacatcggagaccagagagaggaactcaa
    K -
aggggcgctgcctccgggtctggggtcctggcctgcgtggcctgttgg
cacgtgtttctcttcccgcccggcctccagttgtgtgctctcacacag
gcttccttctcgaccggcaggggctggctggcttgcaggccacgaggt
gggctctaccccacactgctttgctgtgtatacgcttgttgccctgaa
ataaatatgcacattttatccatgaaaaaaaaaaaaaaaaaaaa
```

TABLE 5
Aligned Coding and Amino Acid Sequences of the
IgM Autoantibody Light Chain Coding and Protein
Sequences (SEQ ID NOs: 9 and 10, respectively).
The coding sequence extends from nucleotide 13
through 732, excluding the translation
termination codon, in SEQ ID NO: 9.

```
tcaggacacagcatggacatgagggtccccgctcagctcctggggctc
                          M D M R V P A Q L L G L
ctgctgctctggttcccaggttccagatgcgacatccaggaaatagta
  L L L W F P G S R C D I Q E I V
ttgacacagtctccagccaccctgtctgtgtctccaggggaaagagcc
  L T Q S P A T L S V S P G E R A
accctctcctgcagggccagtcagagtgttagcagccacttagcctgg
  T L S C R A S Q S V S S H L A W
taccaacagaaacctggccaggctcccaggctcctcatctatgatgca
  Y Q Q K P G Q A P R L L I Y D A
tccaacagggccactggcatcccagacaggttcagtggcagtgggtct
  S N R A T G I P D R F S G S G S
gggacagacttcactctcaccatcagcagactggagcctgaagatttt
  G T D F T L T I S R L E P E D F
gcagtgtattactgtcagcagtatggtagctcacctccgtacactttc
  A V Y Y C Q Q Y G S S P P Y T F
ggccctgggaccaaagtggatatcaaacgaactgtggctgcaccatct
  G P G T K V D I K R T V A A P S
gtcttcatcttcccgccatctgatgagcagttgaaatctggaactgcc
  V F I F P P S D E Q L K S G T A
tctgttgtgtgcctgctgaataacttctatcccagagaggccaaagta
  S V V C L L N N F Y P R E A K V
cagtggaaggtggataacgccctccaatcgggtaactcccaggagagt
  Q W K V D N A L Q S G N S Q E S
gtcacagagcaggacagcaaggacagcacctacagcctcagcagcacc
  V T E Q D S K D S T Y S L S S T
ctgacgctgagcaaagcagactacgagaaacacaaactctacgcctgc
  L T L S K A D Y E K H K L Y A C
gaagtcacccatcagggcctgagctcgcccgtcacaaagagcttcaac
  E V T H Q G L S S P V T K S F N
aggggagagtgttagagggagaagtgcccccacctgctcctcagttcc
  R G E C -
agcctgaccccctcccatcctttggcctctgaccctttttccacaggg
gacctacccctattgcggtc
```

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu. Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. Antibody vaccines are described in Dillman R. O. (2001) Cancer Invest. 19(8):833-841. Durrant L. G. et al. (2001) Int J. Cancer 1; 92(3):414-20 and Bhattacharya-Chatterjee M, (2001) Curr. Opin. Mol. Ther. February; 3(1):63-9 describe anti-idiotype antibodies. Many of the procedures useful for practicing the present methods, including purification of natural and recombinant antibody molecules. whether or not described herein in detail, are well known to those skilled in the arts of molecular biology, biochemistry, immunology, and medicine.

Monoclonal, polyclonal antibodies, peptide-specific antibodies or single chain recombinant antibodies and antigen binding fragments of any of the foregoing, specifically reacting with the tNOX isoform proteins described herein, may be made by methods known in the art. See e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; Ausubel, F. M. (1990) Current Protocols in Molecular Biology, John Wiley, New York.

Tags, generally located at the N- or C-terminus of a protein of interest, include the polyhistidine sequence (His tag) which allows binding to a nickel or nickel nitriloacetic acid matrix, strep-tag Strep-tag is a synthetic peptide consisting of eight amino acids (Trp-Ser-His-Pro-Gln-Phe-Glu-Lys; SEQ ID NO:11). This peptide sequence exhibits intrinsic affinity towards Strep-Tactin, a specifically engineered streptavidin and can be N- or C-terminally fused to recombinant proteins; a calmodulin-binding peptide fusion system which allows purification using a calmodulin resin; a maltose binding protein fusion system allowing binding to an amylose resin or FLAG tag which contains a known flagellar antigen (Asn-Asp-Tyr-Lys-Asp-Asp-Asp-Asp-Lys-Cys; SEQ ID NO:12).

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The following definitions are provided to clarify their specific use in the context of the invention.

The autoantibody specific to ENOX2 (natural or recombinant) can be incorporated into a pharmaceutical composition for treatment of a cancerous condition in a patient in need thereof, to be administered in an effective amount. Administration can be via any art-known route. Additional therapeutically effective anticancer agents (indium, technetium radioisotopes, Adriamycin, daunomycin, cisplatin and others) can be conjugated to the auto antibody (natural or single chain recombinant) as known to the art. Targeting to cancer cells or tissue is inherent because ENOX2 is expressed on the surface of those cells and tissue.

The exact formulation, route of administration and dosage can be chosen by the individual physician in view of the patient's condition (see, for example, Fingl et al., The Pharmacological Basis of Therapeutics, 1975, Ch. 1 p. 1).

It should be noted that the attending physician would know how to and when to terminate, interrupt, or adjust administration due to toxicity, organ dysfunctions, or other deleterious effects. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents provided herein may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed herein into dosages suitable for systemic administration is within the scope of the invention. With proper choice of carrier and suitable manufacturing practice, the compositions described herein, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds provided herein to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, and then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use as described herein include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions provided herein may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Advantageously, the formulations for parenteral administration are sterile.

The examples provided herein are for illustrative purposes, and are not intended to limit the scope of the invention as claimed herein. Any variations in the exemplified antibodies, epitopes, purification methods, diagnostic methods, preventative methods, treatment methods, and other methods which occur to the skilled artisan are intended to fall within the scope of the present invention.

TABLE 6
Sera from patients with different patterns of ENOX
transcription variants with characteristic molecular
weights and isoelectric points (pH).

| | |
|---|---|
| Transcription | Fully processed |
| variants | remnant |

| Cancer | MW | pH | MW | pH |
|---|---|---|---|---|
| Cervical | 95 | 5.0 | 33 | 5.1 |
| Prostate | 70 | 6.3-6.9 | 38 | 5.4 |
| Breast | 68 | 4.7 | 33 | 4.8 |
| Lung | 54 | 5.1 | 38 | 4.8 |
| Colon | 38 and 52 | 4.4 and 4.1 | 35 | 4.8 |
| Lymph/Leuk | 38-45 | 3.9 | | |
| Ovarian | 40 and 80 | 3.6 and 3.7 | 35 | 5.2 |

TABLE 7
Amino acid sequence (SEQ ID NO: 13) of divalent single chain
autoantibody specific to eNOX2

```
Met Ala Ser Asn Gln Pro Ala Glu Val Gln Leu Val Glu Ser Gly Ala
1 5 10 15
Glu Val Lys Lys Pro Gly Ala Ser Val Lys Val Ser Cys Lys Ala Ser
                        20 25 30
Gly Tyr Thr Phe Thr Ser Tyr Ala Met His Trp Val Arg Gln Ala Pro
                35 40 45
Gly Gln Arg Leu Glu Trp Met Gly Trp Ile Ser Ala Tyr Asn Gly Asn
        50 55 60
Thr Asn Tyr Ala Gln Lys Leu Gln Gly Arg Val Thr Met Thr Thr Val
65 70 75 80
Thr Ser Thr Ser Thr Ala Tyr Met Glu Leu Arg Ser Leu Arg Ser Asp
                                85 90 95
Gly Thr Ala Val Tyr Tyr Cys Ala Arg Asp Leu Leu Val Gly Leu Gly
                        100 105 110
Tyr Trp Gly Gln Gly Thr Leu Val Thr Val Ser Ser Gly Ser Ala Ser
                115 120 125
Ala Pro Thr Leu Phe Pro Leu Val Ser Cys Glu Asn Ser Pro Ser Asp
        130 135 140
Thr Ser Ser Val Ala Val Gly Cys Leu Ala Gln Asp Phe Leu Pro Asp
145 150 155 160
Ser Ile Thr Phe Gly Gly Gly Gly Ser Gly Gly Gly Gly Ser Gly Gly
                                165 170 175
Gly Gly Ser Glu Ile Val Leu Thr Gln Ser Pro Ala Thr Leu Ser Val
                        180 185 190
Ser Pro Gly Glu Arg Ala Thr Leu Ser Cys Arg Ala Ser Gln Ser Val
                195 200 205
Ser Ser His Leu Ala Trp Tyr Gln Gln Lys Pro Gly Gln Ala Pro Arg
        210 215 220
Leu Leu Ile Tyr Asp Ala Ser Asn Arg Ala Thr Gly Ile Pro Asp Arg
225 230 235 240
Phe Ser Gly Ser Gly Ser Gly Thr Asp Phe Thr Leu Thr Ile Ser Arg
                                245 250 255
Leu Glu Pro Glu Asp Phe Ala Val Tyr Tyr Cys Gln Gln Tyr Gly Ser
                        260 265 270
Ser Pro Pro Tyr Thr Phe Gly Pro Gly Thr Lys Val Asp Ile Lys Arg
                275 280 285
Thr Val Ala Ala Pro Ser Val Phe Ile Gly Gly Gly Gly Ser Gly Gly
        290 295 300
Gly Gly Ser Gly Gly Gly Gly Ser Gly Gly Gly Gly Ser Met Ala Ser
305 310 315 320
Asn Gln Pro Ala Glu Val Gln Leu Val Glu Ser Gly Ala Glu Val Lys
                                325 330 335
Lys Pro Gly Ala Ser Val Lys Val Ser Cys Lys Ala Ser Gly Tyr Thr
                        340 345 350
Phe Thr Ser Tyr Ala Met His Trp Val Arg Gln Ala Pro Gly Gln Arg
                355 360 365
Leu Glu Trp Met Gly Trp Ile Ser Ala Tyr Asn Gly Asn Thr Asn Tyr
        370 375 380
Ala Gln Lys Leu Gln Gly Arg Val Thr Met Thr Thr Val Thr Ser Thr
385 390 395 400
Ser Thr Ala Tyr Met Glu Leu Arg Ser Leu Arg Ser Asp Gly Thr Ala
                                405 410 415
Val Tyr Tyr Cys Ala Arg Asp Leu Leu Val Gly Leu Gly Tyr Trp Gly
                        420 425 430
Gln Gly Thr Leu Val Thr Val Ser Ser Gly Ser Ala Ser Ala Pro Thr
                435 440 445
Leu Phe Pro Leu Val Ser Cys Glu Asn Ser Pro Ser Asp Thr Ser Ser
        450 455 460
Val Ala Val Gly Cys Leu Ala Gln Asp Phe Leu Pro Asp Ser Ile Thr
465 470 475 480
Phe Gly Gly Gly Gly Ser Gly Gly Gly Gly Ser Gly Gly Gly Gly Ser
                                485 490 495
Glu Ile Val Leu Thr Gln Ser Pro Ala Thr Leu Ser Val Ser Pro Gly
                        500 505 510
Glu Arg Ala Thr Leu Ser Cys Arg Ala Ser Gln Ser Val Ser Ser His
                515 520 525
Leu Ala Trp Tyr Gln Gln Lys Pro Gly Gln Ala Pro Arg Leu Leu Ile
        530 535 540
Tyr Asp Ala Ser Asn Arg Ala Thr Gly Ile Pro Asp Arg Phe Ser Gly
545 550 555 560
Ser Gly Ser Gly Thr Asp PheThr Leu Thr Ile Ser Arg Leu Glu Pro
                                565 570 575
Glu Asp Phe Ala Val Tyr Tyr Cys Gln Gln Tyr Gly Ser Ser Pro Pro
                        580 585 590
Tyr Thr Phe Gly Pro Gly Thr Lys Val Asp Ile Lys Arg Thr Val Ala
                595 600 605
Ala Pro Ser Val Phe Ile Lys Glu Thr Ala Ala Ala Lys Phe Glu Arg
        610 615 620
Gln His Met Asp Ser
625
```

What is claimed is:

1. An isolated autoantibody which specifically binds to human endogenous ENOX2 as antigen, wherein said autoantibody is characterized by the sequence set forth in SEQ ID NO:6, from amino acid 1 to amino acid 297 or amino acids 1 to 312, or an isolated antibody protein characterized by a heavy chain sequence as set forth in SEQ ID NO:8 and a light chain sequence as set forth in SEQ ID NO:10.

2. The single chain autoantibody of claim 1 comprising a recognition tag.

3. The single chain autoantibody of claim 2, wherein the recognition tag is an S tag, a His tag, a FLAG tag, a Strep tag, a myc tag or a NUS tag and wherein a detectable ligand specifically binds to said tag.

4. A method for detecting cancer-specific ENOX2 isoform proteins in a biological sample, said method comprising the steps of:
a) providing a biological sample;
b) separating the ENOX2 isoform protein(s) from bound autoantibody;
c) reacting the ENOX2 isoform protein(s) separated in step (b) with an isolated autoantibody or a single chain recombinant autoantibody of claim 1 which specifically binds to the ENOX2 isoform proteins; and
d) detecting binding of the autoantibody and ENOX2 isoform proteins,
e) and said method optionally further comprising reacting the ENOX2 isoform proteins separated in step b with at least one other appropriate ENOX2-directed antibody,
whereby a cancer-specific ENOX2 isoform protein is detected in the sample.

5. The method of claim 4, wherein the detecting autoantibody is an autoantibody isolated from cancer patient sera.

6. The method of claim 4, wherein the detecting autoantibody is an isolated ENOX2-specific autoantibody produced in response to ENOX2 antigen.

7. The method of claim 4, wherein the autoantibody is the recombinant autoantibody comprising the amino acid sequence set forth in amino acids 1 to 297 or 1 to 312 of SEQ ID NO:6.

8. The method of claim 5, wherein the method of detection is by means of 2-dimensional polyacrylamide gel electrophoresis and western blotting.

9. The method of claim 4, wherein the step of separating the ENOX2 from its autoantibody is by isoelectric focusing.

10. The method of claim 4, wherein the method of detection is an indirect ELISA.

11. The method of claim 4, wherein the method of detection is a sandwich ELISA.

12. The method of claim 4, wherein the step of detecting is using a pan isoform anti-tNOX single chain variable region (ScFv) autoantibody as a first antibody and a detectable second antibody specific for said first antibody.

13. The method of claim 4, wherein the detectable antibody is detected by enzymatic, chromogenic, chemiluminescent, radiographic, magnetic or fluorescent methods.

14. The method of claim 12, wherein the first antibody is an S-tagged recombinant autoantibody and said method further comprises a step of binding a detectable second anti-S specific antibody or to a ligand bound thereby.

15. The method of claim 12, wherein said detectable second antibody is linked to alkaline phosphatase and wherein binding is detected in the presence of a chromogenic alkaline phosphatase substrate.

16. The method of claim 13, wherein the enzymatic method is a horseradish peroxidase method.

17. The method of claim of claim 4, wherein said biological sample is cells, serum, plasma, or biopsy tissue from a patient suspected of having a neoplastic condition.

18. The method of claim 12, wherein the autoantibody is detected using a second antibody specific for IgM.


SEQUENCE LISTING (23 sequences)

SEQ ID NO: 1 - DNA, 45 bp, Artificial Sequence
  Synthetic constuct  sequence encoding peptide linker
ggaggcggtggatcgggcggtggcggctcgggtggcggcggctct

SEQ ID NO: 2 - protein, 15 aa, Artificial Sequence
  Synthetic construct  linker peptide
GGGGSGGGGSGGGGS

SEQ ID NO: 3 - DNA, 45 bp, Artificial Sequence
  Synthetic construct  sequence encoding S tag
aaagaaaccgctgctgctaaattcgaacgccagcacatggacagc

SEQ ID NO: 4 - protein, 15 aa, Artificial Sequence
  Synthetic construct  S tag
KETAAAKFERQHMDS

SEQ ID NO: 5 - DNA, 939 bp, Artificial Sequence
  Synthetic construct  sequence encoding single chain antibody
atggctagcaatcagccggcagaggtgcagctggtggagtctggggctgaggtgaagaagcctggggcctcagtgaaggtttcctgcaaggcttctggatacaccttcactagctatgctatgcattgggtgcgccaggcccccggacaaaggcttgagtggatgggatggatcagcgcttacaatggtaacacaaactatgcacagaagctccagggcagagtcaccatgaccacagtcacatccacgagcacagcctacatggagctgaggagcctgagatctgacggcacggccgtgtattactgtgcgagagatctgctagtgggcctcggctactggggccagggaaccctggtcaccgtctcctcagggagtgcatccgccccaacccttttccccctcgtctcctgtgagaattccccgtcggatacgagcagcgtggccgttggctgcctcgcacaggacttccttcccgactccatcactttcggaggcggtggatcgggcggtggcggctcgggtggcggcggctctgaaatagtattgacacagtctccagccaccctgtctgtgtctccaggggaaagagccaccctctcctgcagggccagtcagagtgttagcagccacttagcctggtaccaacagaaacctggccaggctcccaggctcctcatctatgatgcatccaacagggccactggcatcccagacaggttcagtggcagtgggtctgggacagacttcactctcaccatcagcagactggagcctgaagattttgcagtgtattactgtcagcagtatggtagctcacctccgtacactttcggccctgggaccaaagtggatatcaaacgaactgtggctgcaccatctgtcttcatcaaagaaaccgcagctgctaaattcgaacgccagcacatggacagttaa

SEQ ID NO: 6 - protein, 312 aa, Artificial Sequence
  Synthetic construct  single chain antibody
MASNQPAEVQLVESGAEVKKPGASVKVSCKASGYTFTSYAMHWVRQAPGQRLEWMGWISAYNGNTNYAQKLQGRVTMTTVTSTSTAYMELRSLRSDGTAVYYCARDLLVGLGYWGQGTLVTVSSGSASAPTLFPLVSCENSPSDTSSVAVGCLAQDFLPDSITFGGGGSGGGGSGGGGSEIVLTQSPATLSVSPGERATLSCRASQSVSSHLAWYQQKPGQAPRLLIYDASNRATGIPDRFSGSGSGTDFTLTISRLEPEDFAVYYCQQYGSSPPYTFGPGTKVDIKRTVAAPSVFIKETAAAKFERQHMDS

SEQ ID NO: 7 - DNA, 2108 bp, Homo sapiens
ttttgtttaactttaagaaggagatatacatatggctagcaatcagccggcagaggtgcagctggtggagtctggggctgaggtgaagaagcctggggcctcagtgaaggtttcctgcaaggcttctggatacaccttcactagctatgctatgcattgggtgcgccaggcccccggacaaaggcttgagtggatgggatggatcagcgcttacaatggtaacacaaactatgcacagaagctccagggcagagtcaccatgaccacagtcacatccacgagcacagcctacatggagctgaggagcctgagatctgacggcacggccgtgtattactgtgcgagagatctgctagtgggcctcggctactggggccagggaaccctggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcagcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgatcccaacagaagaacatcggagaccagagagaggaactcaaaggggcgctgcctccgggtctggggtcctggcctgcgtggcctgttggcacgtgtttctcttcccgcccggcctccagttgtgtgctctcacacaggcttccttctcgaccggcaggggctggctggcttgcaggccacgaggtgggctctaccccacactgctttgctgtgtatacgcttgttgccctgaaataaatatgcacattttatccatgaaaaaaaaaaaaaaaaaaaa

SEQ ID NO: 8 - protein, 599 aa, Homo sapiens
MASNQPAEVQLVESGAEVKKPGASVKVSCKASGYTFTSYAMHWVRQAPGQRLEWMGWISAYNGNTNYAQKLQGRVTMTTVTSTSTAYMELRSLRSDGTAVYYCARDLLVGLGYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEGEVSADEEGFENLWATASTFIVLFLLSLFYSTTVTLFKVK

SEQ ID NO: 9 - DNA, 836 bp, Homo sapiens
tcaggacacagcatggacatgagggtccccgctcagctcctggggctcctgctgctctggttcccaggttccagatgcgacatccaggaaatagtattgacacagtctccagccaccctgtctgtgtctccaggggaaagagccaccctctcctgcagggccagtcagagtgttagcagccacttagcctggtaccaacagaaacctggccaggctcccaggctcctcatctatgatgcatccaacagggccactggcatcccagacaggttcagtggcagtgggtctgggacagacttcactctcaccatcagcagactggagcctgaagattttgcagtgtattactgtcagcagtatggtagctcacctccgtacactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtc

SEQ ID NO: 10 - protein, 240 aa, Homo sapiens
MDMRVPAQLLGLLLLWFPGSRCDIQEIVLTQSPATLSVSPGERATLSCRASQSVSSHLAWYQQKPGQAPRLLIYDASNRATGIPDRFSGSGSGTDFTLTISRLEPEDFAVYYCQQYGSSPPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 11 - protein, 8 aa, Artificial Sequence
  Synthetic construct  Strep tag sequence
WSHPQFEK

SEQ ID NO: 12 - protein, 10 aa, Artificial Sequence
  Synthetic construct  FLAG tag sequence
NDYKDDDDKC

SEQ ID NO: 13 - protein, 629 aa, Artificial Sequence
  Synthetic construct  tandem repeat of single chain antibody with intervening linker peptide sequence
MASNQPAEVQLVESGAEVKKPGASVKVSCKASGYTFTSYAMHWVRQAPGQRLEWMGWISAYNGNTNYAQKLQGRVTMTTVTSTSTAYMELRSLRSDGTAVYYCARDLLVGLGYWGQGTLVTVSSGSASAPTLFPLVSCENSPSDTSSVAVGCLAQDFLPDSITFGGGGSGGGGSGGGGSEIVLTQSPATLSVSPGERATLSCRASQSVSSHLAWYQQKPGQAPRLLIYDASNRATGIPDRFSGSGSGTDFTLTISRLEPEDFAVYYCQQYGSSPPYTFGPGTKVDIKRTVAAPSVFIGGGGSGGGGSGGGGSGGGGSMASNQPAEVQLVESGAEVKKPGASVKVSCKASGYTFTSYAMHWVRQAPGQRLEWMGWISAYNGNTNYAQKLQGRVTMTTVTSTSTAYMELRSLRSDGTAVYYCARDLLVGLGYWGQGTLVTVSSGSASAPTLFPLVSCENSPSDTSSVAVGCLAQDFLPDSITFGGGGSGGGGSGGGGSEIVLTQSPATLSVSPGERATLSCRASQSVSSHLAWYQQKPGQAPRLLIYDASNRATGIPDRFSGSGSGTDFTLTISRLEPEDFAVYYCQQYGSSPPYTFGPGTKVDIKRTVAAPSVFIKETAAAKFERQHMDS

SEQ ID NO: 14 - DNA, 23 bp, Artificial Sequence
  Synthetic construct  primer
saggtgmagytgktggagtctgg

SEQ ID NO: 15 - DNA, 24 bp, Artificial Sequence
  Synthetic construct  primer
aagtgatggagtcgggaaggaagt

SEQ ID NO: 16 - DNA, 24 bp, Artificial Sequence
  Synthetic construct  primer
gaaatwgtrwtgacrcagtctcca

SEQ ID NO: 17 - DNA, 24 bp, Artificial Sequence
  Synthetic construct  primer
gatgaagacagatggtgcagccac

SEQ ID NO: 18 - DNA, 28 bp, Artificial Sequence
  Synthetic construct  primer
atatcagccggccsaggtgmagytgktg

SEQ ID NO: 19 - DNA, 62 bp, Artificial Sequence
  Synthetic construct  primer
agagccgccgccacccgagccgccaccgcccgatccaccgcctccgaaagtgatggagtcgg

SEQ ID NO: 20 - DNA, 69 bp, Artificial Sequence
  Synthetic construct  primer
ggaggcggtggatcgggcggtggcggctcgggtggcggcggctctgaaatwgtrwtgacrcagtctcca

SEQ ID NO: 21 - DNA, 33 bp, Artificial Sequence
  Synthetic construct  primer
agcggccgcgatgaagacagatggtgcagccac

SEQ ID NO: 22 - DNA, 35 bp, Artificial Sequence
  Synthetic construct  primer
catatacatatggctagcaatcagccggccsaggt

SEQ ID NO: 23 - DNA, 80 bp, Artificial Sequence
  Synthetic construct  primer
tagcatccggatccttaactgtccatgtgctggcgttcgaatttagcagctgcggtttctttgatgaagacagatggtgc